(19) United States
(12) Patent Application Publication
(10) Pub. No.: US 2011/0072563 A1
(43) Pub. Date: Mar. 31, 2011

(54) PIN LOCK DEVICE AND HELMET INCLUDING SAME

(75) Inventors: Kwang Moon Choi, Gyeonggi-do (KR); Young Il Kim, Gyeonggi-do (KR)

(73) Assignee: HJC CORP., Gyeonggi-do (KR)

(21) Appl. No.: 12/880,680

(22) Filed: Sep. 13, 2010

(30) Foreign Application Priority Data
Sep. 29, 2009 (KR) 10-2009-0092270

(51) Int. Cl. A42B 3/04; F16B 7/20; A61F 9/00

(52) U.S. Cl. 2/422; 403/349; 2/15

(57) ABSTRACT

The present disclosure relates to a pin lock device allowing detachable attachment of an anti-moisture film or a shield protective film to a surface of a shield secured to a helmet main body in an openable/closable manner and a helmet including same.

### FIELD OF THE INVENTION

The present disclosure relates to a pin lock device allowing detachable attachment of a film to a surface of a shield for use in a helmet and a helmet including same; and, more particularly, to a pin lock device enabling attachment of a shield protective film and an anti-moisture film to a shield while allowing an increase of the lifetime of the shield and comfortable use of a helmet, and also relates to a helmet including such a pin lock device.

### BACKGROUND OF THE INVENTION

Wearing a helmet is required to drive a two-wheeled vehicle such as a motorcycle. An opening is provided in the front portion of a helmet main body to secure visibility ahead of a driver, and an openable/closable shield is positioned at the front opening of the helmet main body.

Since the helmet has a sealed structure lacking sufficient ventilation between the inside and the outside thereof, the driver may feel stuffy, and the inside of the helmet may get damp as the driver breathes, obstructing the driver's field of vision. To solve the problem of generation of moisture inside the shield, there has been provided a helmet having an anti-moisture film attached to an inner side of the shield.

Typically, the shield exposed to the outside of the helmet is generally made of plastic or the like to secure visibility and to allow easy opening/closing operation. In such a case, however, if a damage or a scratch is inflicted on the surface of the shield due to impurities from the outside, there has been known a technique to replace the shield or to attach a shield protective film to the outside of the shield to obtain visibility.

### BRIEF SUMMARY OF THE INVENTION

In view of the foregoing, the present disclosure provides an improved pin lock device secured to a shield and capable of allowing easy attachment of an anti-moisture film or a shield protective film to the inside or the outside of the shield while allowing easy replacement of the films. Further, the present disclosure also provides an improved helmet having such a pin lock device.

In accordance with one aspect of the present disclosure, there is provided a pin lock device of a shield secured to a helmet main body in an openable/closable manner, the pin lock device including: lock insertion holes, formed through both lateral surfaces of the shield; lock pins, each being inserted into each of the lock insertion holes from the inside of each lock insertion hole toward the outside thereof; and lock bases, each being engaged with each of the lock pins from the outside of each lock insertion hole. The each lock pin includes: a pin protrusion to be fitted into each of the lock bases through each of the lock insertion holes in an outward direction; a pin supporting plate to be brought into firm contact with each of the lock insertion holes at an inner side of the shield to stop an outward movement of each lock pin; and a pin handle inwardly extending from the pin supporting plate and serving as a head of each lock pin.

Further, each of the lock pins may further includes: each of pin grooves formed as a curved cut portion between the pin supporting plate and the pin handle and having a cross section smaller than cross sections of the pin supporting plate and the pin handle. An anti-moisture film may be detachably fitted between the pin grooves along both inner lateral surfaces of the shield.

Further, each lock base may includes: a base protrusion to be accommodated in each of the lock insertion holes while allowing each lock pin to be inserted into a cavity formed within the base protrusion; a base supporting plate to be brought into firm contact with each of the lock insertion holes at an outer side of the shield to stop an inward movement of each lock base; and a base handle outwardly extending from the base supporting plate and serving as a head of each lock base.

Moreover, each of the lock bases may further includes: each of base grooves formed as a curved cut portion between the base supporting plate and the base handle and having a cross section smaller than cross sections of the base supporting plate and the base handle. A shield protective film may be detachably fitted between the base grooves along both outer lateral surfaces of the shield.

In addition, each lock base may further includes: a protrusion accommodating part formed within each of the lock bases to be positioned around the cavity to prevent separation of each lock pin from each lock base. The cavity may be formed in a shape corresponding to a shape of the pin protrusion. If the pin protrusion inserted in the cavity rotates within each of the lock bases in a secured state, the pin protrusion may come out of the cavity and may be positioned on the protrusion accommodation part, and, thus, each lock pin is fixed in place.

Further, both the cavity and the pin protrusion may have straight line shapes.

Furthermore, the base protrusion may be formed to be located at an eccentric position of each of the lock bases, and a distance between the lock bases at both lateral surfaces of the shield is varied when each of the lock bases rotates while engaged with each lock pin.

In accordance with another aspect of the present disclosure, there is provided a helmet including a shield secured to a helmet main body in an openable/closable manner and pin lock devices fastened to both lateral surfaces of the shield, the helmet including: lock insertion holes, formed through both lateral surfaces of the shield; lock pins, each being inserted into each of the lock insertion holes from the inside of each lock insertion hole toward the outside thereof; and lock bases, each being engaged with each of the lock pins from the outside of each lock insertion hole. Each lock pin includes: a pin protrusion to be fitted into each of the lock bases through each of the lock insertion holes in an outward direction; a pin supporting plate to be brought into firm contact with each of the lock insertion holes at an inner side of the shield to stop an outward movement of each lock pin; and a pin handle inwardly extending from the pin supporting plate and serving as a head of each lock pin. Each lock base includes: a base protrusion to be accommodated in each of the lock insertion holes while allowing each lock pin to be inserted into a cavity formed within the base protrusion; a base supporting plate to be brought into firm contact with each of the lock insertion holes at an outer side of the shield to stop an inward movement of each lock base; and a base handle outwardly extending from the base supporting plate and serving as a head of each lock base.

Further, each of the lock pins may further includes each of pin grooves formed as a curved cut portion between the pin supporting plate and the pin handle and having a cross section smaller than cross sections of the pin supporting plate and the pin handle. An anti-moisture film may be detachably fitted between the pin grooves along both inner lateral surfaces of the shield. Each of the lock bases may further includes each of base grooves formed as a curved cut portion between the base supporting plate and the base handle and having a cross section smaller than cross sections of the base supporting plate and the base handle. A shield protective film may be detachably fitted between the base grooves along both outer lateral surfaces of the shield.

Furthermore, each lock base may further includes a protrusion accommodating part formed within each of the lock bases to be positioned around the cavity to prevent separation of each lock pin from each lock base. The cavity may be formed in a shape corresponding to a shape of the pin protrusion. If the pin protrusion inserted in the cavity rotates within each of the lock bases in a secured state, the pin protrusion may come out of the cavity and may be positioned on the protrusion accommodation part, and, thus, each lock pin is fixed in place.

Moreover, both the cavity and the pin protrusion may have straight line shapes.

Furthermore, the base protrusion may be formed to be located at an eccentric position of each of the lock bases. A distance between the lock bases at both lateral surfaces of the shield may be varied when each of the lock bases rotates while engaged with each lock pin.

In accordance with the present disclosure as described above, it is possible to provide an improved pin lock device secured to a shield and capable of allowing easy attachment of an anti-moisture film or a shield protective film to the inside or the outside of the shield while allowing easy replacement thereof. Further, an improved helmet having the pin lock device can also be provided.

### BRIEF DESCRIPTION OF THE DRAWINGS

Non-limiting and non-exhaustive embodiments will be described in conjunction with the accompanying drawings. Understanding that these drawings depict only several embodiments in accordance with the disclosure and are, therefore, not to be intended to limit its scope, the disclosure will be described with specificity and detail through use of the accompanying drawings, in which:

FIG. 1 is a perspective view of a helmet in accordance with an embodiment of the present disclosure;

FIG. 2 is an exploded perspective view of a shield, a pin lock device, an anti-moisture film and a shield protective film in accordance with the embodiment of the present disclosure;

FIGS. 3A and 3B illustrate a state in which a lock pin is engaged with a lock base with the shield positioned therebetween: FIG. 3A shows a state in which the lock pin is fitted into a center position of the lock base while FIG. 3B shows a state in which the lock pin is fitted into an eccentric position of the lock base;

FIG. 4 illustrates a state in which the lock base is engaged with the lock pin with the shield positioned therebetween;

FIGS. 5A and 5B illustrate a state in which the lock base engaged with the lock base through a lock insertion hole rotates about a base protrusion provided at an eccentric position of the lock base: FIG. 5A shows a state in which a center of the base protrusion is positioned outside a center of the lock base while FIG. 5B shows a state in which the center of the base protrusion is positioned inside the center of the lock base;

FIGS. 6A and 6B are cross sectional views illustrating a state before the lock pin is engaged with the lock base: FIG. 6A shows a state in which the lock pin is fitted into a center position of the lock base while FIG. 6B shows a state in which the lock pin is fitted into an eccentric position of the lock base;

FIG. 7 provides a cross sectional view showing a state immediately after the lock pin is inserted into the lock base and also provides a partial enlarged view of an inserted part; and

FIG. 8 provides a cross sectional view showing a state in which the lock pin inserted into the lock base is firmly engaged with the lock base through rotation and also provides a partial enlarged view showing an engaged part.

### DETAILED DESCRIPTION OF THE INVENTION

Hereinafter, embodiments of the present invention will be described in detail with reference to the accompanying drawings so that the present invention may be readily implemented by those skilled in the art. However, it is to be noted that the present invention is not limited to the embodiments but can be realized in various other ways. In the drawings, parts irrelevant to the description are omitted for the simplicity of explanation, and like reference numerals denote like parts through the whole document.

Further, the term “comprises or includes” and/or “comprising or including” used in the document does not mean that existence or addition of one or more other components or elements is excluded in addition to the described components or elements unless otherwise specified.

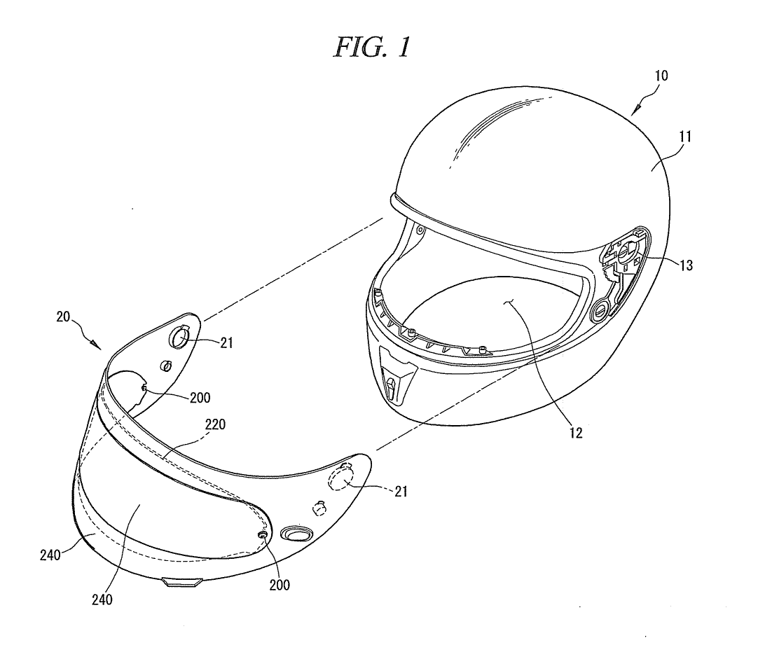

Hereinafter, a pin lock device and a helmet having the pin lock device in accordance with an embodiment of the present disclosure will be explained with reference to FIGS. 1 to 8.

FIG. 1 is a perspective view of a helmet 10 in accordance with the embodiment of the present disclosure.

As illustrated in FIG. 1, the helmet 10 in accordance with the present embodiment includes a helmet main body 11, an opening 12 and a shield 20 secured to the helmet main body 11 to be opened or closed.

The helmet main body 11 serves as a main body of the helmet 10, and it has an inner space in which a user's head is accommodated. To firmly fit the users' head, the helmet main body 10 is designed to have a sealed structure. The helmet main body 11 is provided with the opening 12 at its front surface to secure visibility ahead of it.

The shield 20 is a transparent window capable of selectively opening and closing the opening 12 lest the field of vision should be obstructed by wind, rain, snow or the like blown from ahead when a vehicle travels. Inner engagement members 21 capable of being engaged with the helmet main body 11 are formed at both lateral end surfaces of the shield 20. The shield 20 is secured to the helmet main body 11 as shield engagement members 13 formed along both lateral surfaces of the helmet main body 11 are engaged with the inner engagement members 21.

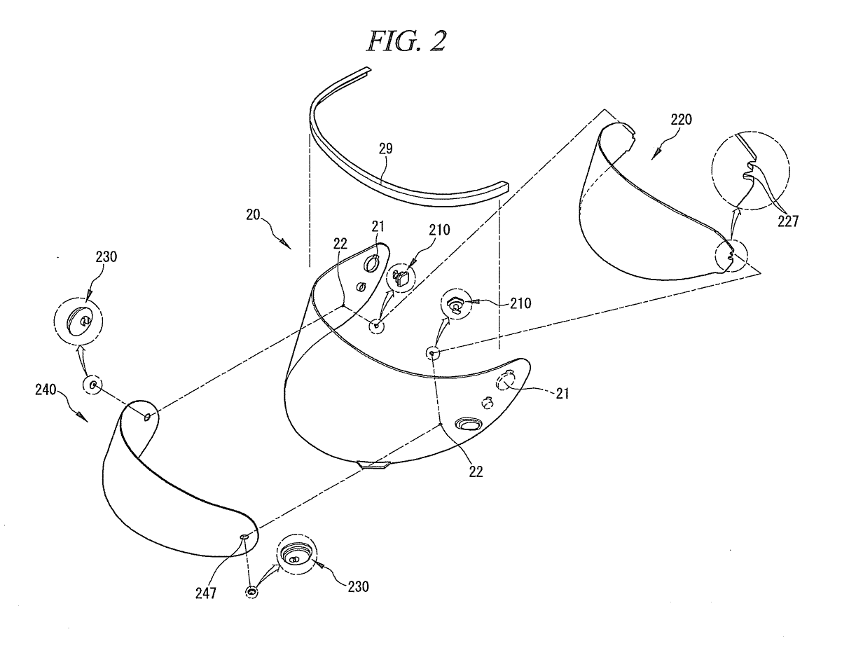

The shield 20 may include lock insertion holes 22 formed through both lateral surfaces thereof (see FIG. 2). Pin lock devices 200 can be secured to the shield 20 as they are fitted into the lock insertion holes 22. Further, at the inner side of the shield 20, an anti-moisture film 220 can be insertion-fitted between the pin lock devices 200 secured to both lateral surfaces of the shield 20. Furthermore, at the outer side of the shield 20, a shield protective film 240 can be fitted between the pin lock devices 200 secured to both lateral surfaces of the shield 20. The shield 20, the pin lock devices 200 and the films 220 and 240 will be described in further detail with reference to FIGS. 2 to 4.

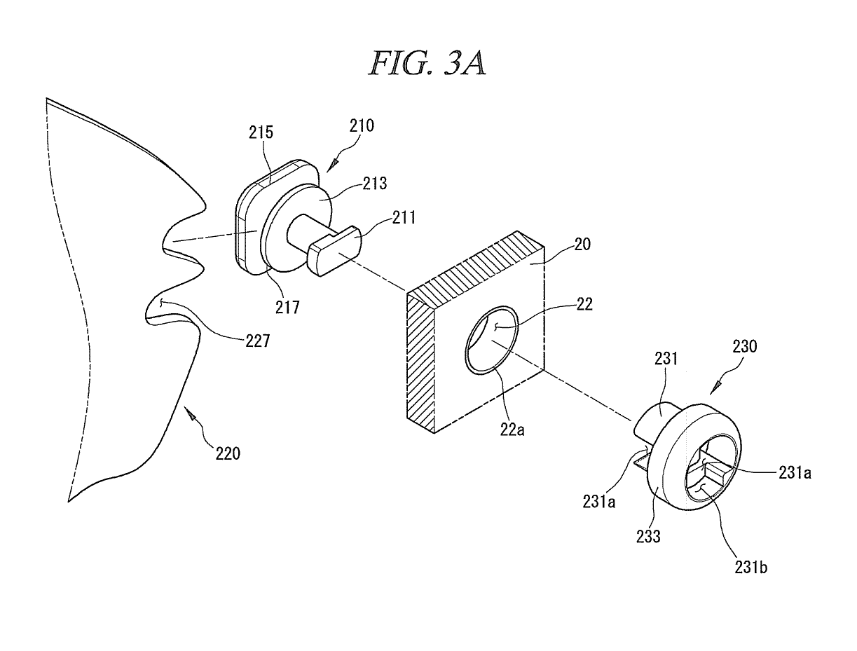

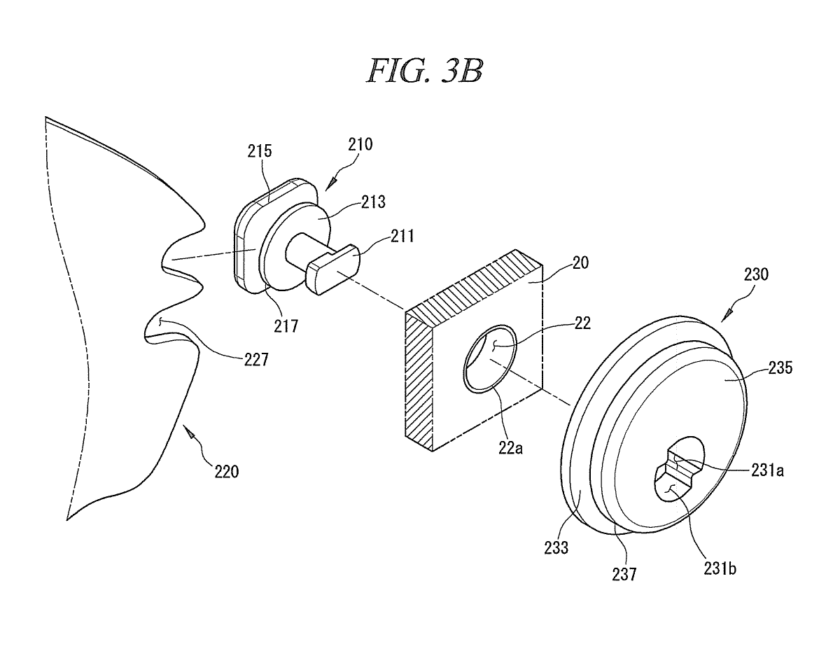

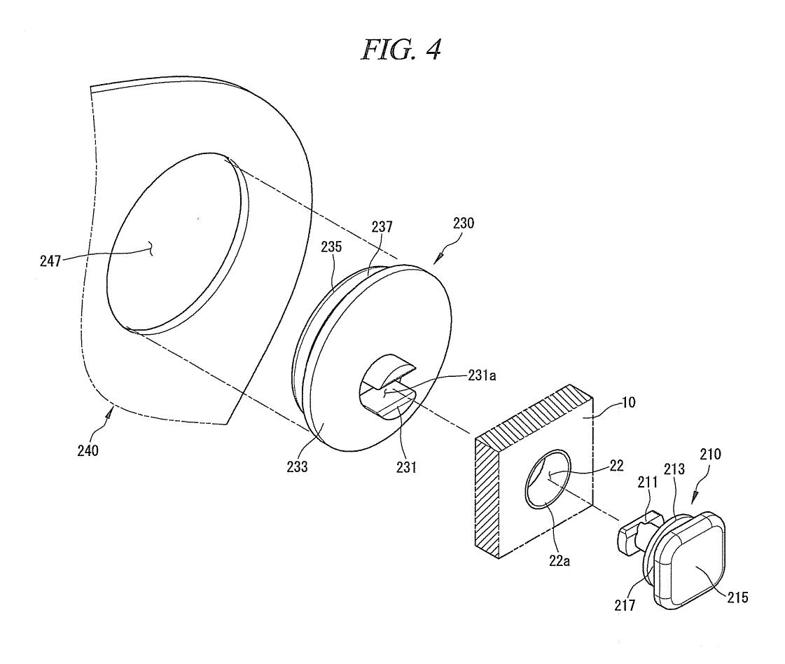

FIG. 2 is an exploded perspective view illustrating the shield 20, the pin lock devices 200, the anti-moisture film 220 and the shield protective film 240. FIG. 3 shows a state in which a lock pin 210 is engaged with a lock base 230 with the shield 20 positioned therebetween, and the anti-moisture film 220 can be detachably insertion-fitted between them. Here, as illustrated in FIGS. 3A and 3B, the lock base 230 may have various shapes as long as it can be engaged with the lock pin 210 while accommodated on the shield 20. FIG. 4 illustrates a state in which the lock base 230 is engaged with the lock pin 210 with the shield 20 interposed therebetween, and the shield protective film 240 can be detachably fitted to the lock base 230.

As shown in FIG. 2, the shield 20 is provided with the lock insertion holes 22 formed through its both lateral surfaces. The pin lock devices 200 are fitted in these lock insertion holes 22.

Each lock insertion hole 22 has a diameter capable of accommodating therein a pin protrusion 211 of the pin lock device 200 inserted therethrough while allowing the pin protrusion 211 accommodated in a base protrusion 231 to be rotated in clockwise direction or counterclockwise direction. The lock insertion hole 22 is formed in each of both lateral surfaces of the shield 20, and it may be provided with a bearing 22a (see FIGS. 3A and 3B).

The bearing 22a is a ring-shaped member formed at the inside of the pin insertion hole 22 to conform to the outline of the pin insertion hole 22. When the pin lock device 200 is rotated after it is secured to the shield 20, the bearing 22a may absorb friction, facilitating smooth rotation.

Furthermore, the shield 20 may include an elastic member 29 that may absorb impact applied to the shield 20 from the collision with the helmet main body 11 when the shield 20 is inserted into the inside of the helmet 10. The elastic member 29 may be made of an elastic material such as rubber and can be insertion-fitted in an upper portion of the shield 20.

The pin lock devices 200 are coupling members that are secured to the shield 20 by being fitted in the lock insertion holes 22 provided in both lateral surfaces of the shield 20. The pin lock devices 200 support the films 220 and 240 to be described later in detachable manner. For the purpose, each pin lock device 200 may include the lock pin 210 and the lock base 230.

The lock pin 210 is a coupling member inserted from the inside of the lock insertion hole 22 to the outside thereof. The lock pin 210 may include a pin protrusion 211, a pin supporting plate 213 and a pin handle 215.

The pin protrusion 211 is a protrusion outwardly fitted into the lock base 230 through the lock insertion hole 22.

The pin supporting plate 213 is a protruding member firmly in contact with the lock insertion hole 22 at the inner side of the shield 20 to stop further outward movement of the lock pin 210.

The pin handle 215 is a handle of the lock pin 210, and it is extended inward from the pin supporting plate 213 and is configured as a head of the lock pin 210.

Further, the lock pin 210 may further include a pin groove 217.

The pin groove 217 is a curved cut portion formed between the pin supporting plate 213 and the pin handle 215 and having a cross section smaller than cross sections of the pin supporting plate 213 and the pin handle 215.

As illustrated in FIGS. 3A and 3B, the lock pin 210 is engaged with the lock base 230 with the shield 20 positioned therebetween. The anti-moisture film 220 can be detachably fitted to the lock pin 210.

The anti-moisture film 220 is secured to the inner side of the shield 20 and prevents the shield 20 to become foggy due to moisture generated when the user of the helmet 10 breathes. The anti-moisture film 220 may be provided with engagement grooves 227 along both end portions thereof.

As shown in FIGS. 3A and 3B, each engagement groove 227 may be formed in a wave shape and is fitted to the pin groove 217 formed on the lock pin 210.

The anti-moisture film 220 is secured to the shield 20 by engaging the engagement grooves 227 formed at both ends of the anti-moisture film 220 with the pin grooves 217 of the lock pins 210 inserted in both lateral surfaces of the shield 20. At this time, since the anti-moisture film 220 is bent or deformed, it can be secured to or separated from the inner side of the shield 20 without having to separate the lock pins 210 or the like. Here, FIGS. 3A and 3B only shows an example of the engagement grooves 227, and the engagement grooves 227 may have various other shapes and cross sections as long as they can be engaged with the pin grooves 217. Furthermore, the anti-moisture film 220 may be made of such a material as plastic having a light shielding effect so as to protect the driver's eyes by blocking sun light during day time, thus allowing sufficient field of vision even under strong sun light or reflection light.

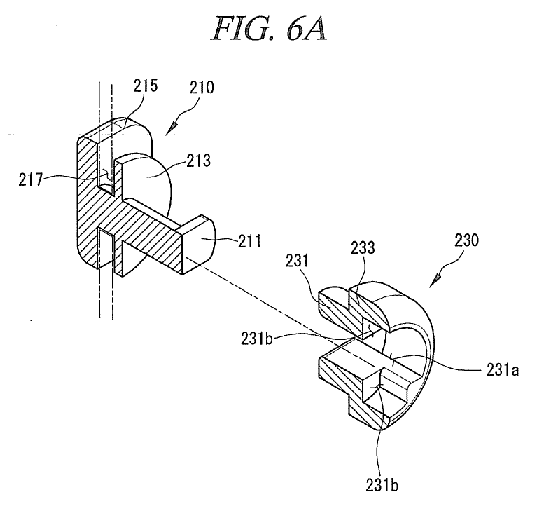

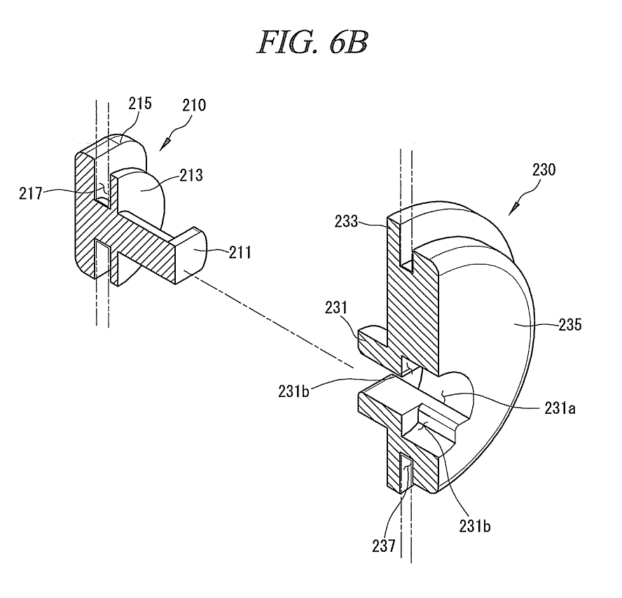

As shown in FIG. 3A, the lock base 230 is a coupling member engaged with the lock pin 210 from the outside of the lock insertion hole 22. The lock base 230 may include a base protrusion 231 formed at a center (center position, see FIG. 6A for more detail) of the lock base 230; a cavity 231a; a protrusion accommodating part 231b; and a base supporting plate 233.

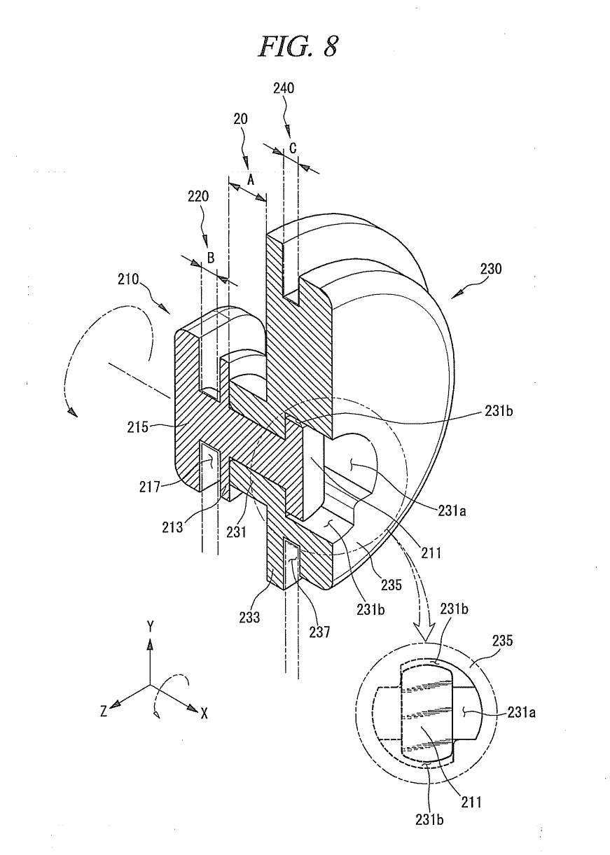

The base protrusion 231 is a protrusion to be accommodated in the lock insertion hole 22 and having therein the cavity 231a into which the lock pin 210 is inserted. The cavity 231a may be formed in a straight line shape, and the pin protrusion 211 inserted into the cavity 231a may have a straight line shape corresponding thereto. That is, the cavity 231a and the pin protrusion 211 are formed in mutually corresponding shapes. Accordingly, when the pin protrusion 211 inserted in the cavity 231a is rotated within the lock base 230 in a secured state, the pin protrusion 211 comes out of the cavity 231a and becomes positioned on the protrusion accommodating part 231b, allowing the lock pin 210 to be fixed in place (see FIG. 8 for more detail).

The cavity 231a extending from the inside of the base protrusion 231 may be formed through the lock base 230. Further, the protrusion accommodating part 231b may be formed around the cavity 231a (see FIG. 8 for more detail).

The protrusion accommodating part 231b is an inner region of the lock base 230 formed around the cavity 231a and blocked in an inward direction of the shield 20 so as to prevent unintended separation of the lock pin 210 which is inserted into and engaged with the lock base 230. Accordingly, if the lock pin 210 inserted into the lock base 230 comes out of the cavity 231a by rotation and is positioned on the protrusion accommodating part 231b (see FIG. 8 for more detail), unintended separation of the lock pin 210 from the lock base 230 can be avoided. Thus, in the state that lock pin 210 or the lock base 230 are secured to the shield 20, the user of the helmet 10 can still rotate the lock pin 210 or the lock base 230 freely in clockwise direction or counterclockwise direction. Meanwhile, to separate the lock pin 210 from the lock base 230, the user of the helmet 10 may pull the lock pin 21 toward the inner side of the shield 20, thus allowing the lock pin 210 to be separated and removed from the lock base 230.

The base supporting plate 233 is a protruding member firmly in contact with the lock insertion hole 22 from the outer side of the shield 20 to stop inward movement of the lock base 230.

Further, the lock base 230 may include, as illustrated in FIG. 3B, a base protrusion 231 protrudently formed at an eccentric position (see FIG. 6B for more detail), a base supporting plate 233 and a base handle 235. The base protrusion 231 protrudently formed at the eccentric position is a protrusion protrudently formed in a direction toward the shield 20 at a position deviated from a center of the lock base 230.

The base handle 235 is a handle of the lock base 230, and it is extended outward from the base supporting plate 233 and is configured as a head of the lock base 230. The base handle 235 may be useful when the user of the helmet 10 rotates the lock base 230 secured to the shield 20 in clockwise direction or counterclockwise direction.

Furthermore, the lock base 230 may further include a base groove 237.

The base groove 237 is a curved cut portion formed between the base supporting plate 233 and the base handle 235 and having a cross section smaller than cross sections of the base supporting plate 233 and the base handle 235.

As illustrated in FIG. 4, the lock base 230 is engaged with the lock pin 210 with the shield 20 positioned therebetween. The shield protective film 240 can be detachably fitted to the lock base 230.

The shield protective film 240 is secured to the outer side of the shield 20 and protects the shield 20 so as to prevent generation of, e.g., scratch on the shield 20 even in case an impact is applied to the shield 20 from the outside of the helmet 10 or the shield 20 collides with an external object. Further, the shield protective film 240 can prevent contamination of the shield 20 with dirt or the like. By replacing the shied protective film 240, clean field of vision can be secured continuously. Further, the shield protective film 240 may have engagement grooves 247 at both end portions thereof.

As illustrated in FIG. 4, the engagement grooves 247 may be formed at both end portions of the shield protective film 240. As the engagement groove 247 is engaged with the base groove 237 formed at the lock base 230, the shield protective film 240 can be secured to the lock base 230.

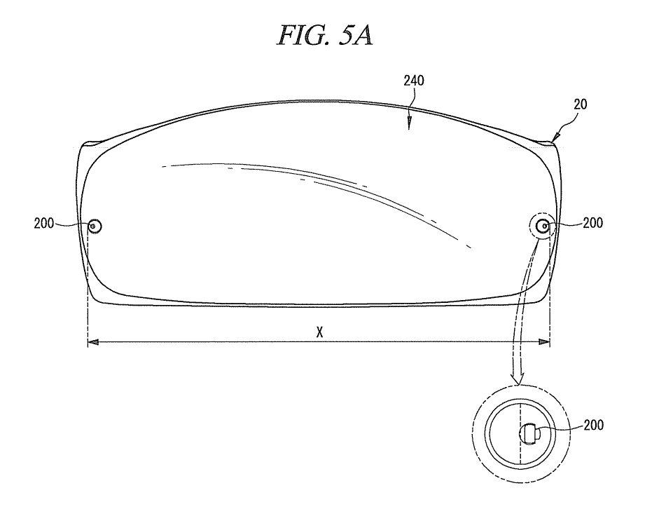

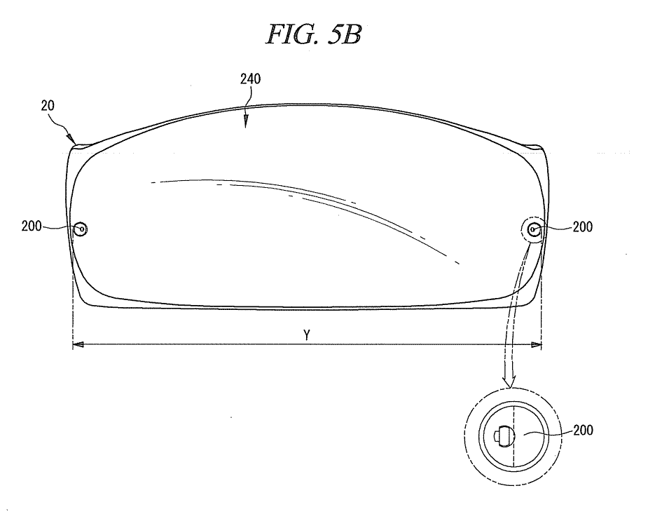

Below, a principle for adjusting a distance between the lock bases 230 by the rotation of the lock bases 230 will be described with reference to FIGS. 5A and 5B, wherein the lock bases 230 are secured to the shield 20 with eccentric structure (eccentric position).

FIGS. 5A and 5B show a state in which the lock base 230 engaged with the lock pin 210 through the lock insertion hole 22 rotates about the base protrusion 231 located at an eccentric position. FIG. 5A shows a state in which a center of the base protrusion 231 is positioned outside a center of the lock base 230, and FIG. 5B shows a state in which the center of the base protrusion 231 is positioned inside the center of the lock base 230.

As illustrated in FIGS. 5A and 5B, the center of the lock base 230 varies depending on the rotation of the lock base 230, whereas the center of the base protrusion 231 is fixed since the base protrusion 231 is rotated while it is secured to the lock pin 210. Accordingly, in case that the base protrusion 231 is located at the eccentric position of the lock base 230, the distance between the lock bases 230 secured to both lateral surfaces of the shield 20 is varied as the lock base 230 rotates.

In case that the centers of the base protrusions 231 located at both lateral sides of the shield 20 are positioned outside the centers of the lock base 230 (FIG. 5), a distance X between the lock bases 230 may be shortened as compared to a case in which the base protrusions 231 are located at the centers of the lock bases 230. Further, in case that the centers of the base protrusions 231 are positioned inside the centers of the lock bases 230 (FIG. 5B), a distance between the lock bases 230 may be increased as compared to a case in which the base protrusions 231 are located at the centers of the lock bases 230. Accordingly, Y is larger than X. That is, the distance between the lock bases 230 increases if the base protrusions 231 in the eccentric positions are located inside the centers of the lock bases 230, while the distance between the lock bases 230 decreases if the base protrusions 231 in the eccentric positions are located outside the centers of the lock bases 230.

Accordingly, the shield protective film 240 fitted to the base groove 237 as the curved cut portion of the lock base 230 can be tightened or loosened so as to be separated from the base groove 237 depending on the variable distance between the lock bases 230 secured to both lateral surfaces of the shield 20. Accordingly, when the shield protective film 240 needs to be replaced, the shield protective film 230 can be easily removed from the shield 20 by rotating the lock bases 230 so that the distance between the lock bases 230 is shortened. Further, the shield protective film 240 can be made to come into firm contact with the shield 20 by rotating the lock bases 230 such that the distance between them increases. For the purpose, the shield protective film 240 may be formed of a transparent elastic member.

Hereinafter, engagement of the lock pin 210 with the lock base 230 will be described with reference to FIGS. 6A to 8.

FIGS. 6A and 6B are cross sectional views illustrating the lock pin 210 and the lock base 230 before they are engaged with each other. FIG. 6A shows a state in which the lock pin 210 is fitted into a center position of the lock base 230, and FIG. 6B shows a state in which the lock pin 210 is fitted into an eccentric position of the lock base 230.

As shown in FIGS. 6A and 6B, the lock pin 210 has the pin protrusion 211 capable of being fitted into the lock base 230. Further, the lock pin 210 may include the pin supporting plate 213, the pin handle 215 and the pin groove 217.

The lock base 230 is a coupling member into which the lock pin 210 can be inserted through the cavity 231a formed at the inside of the lock base 230. The lock base 230 may include the base protrusion 231, the base supporting plate 233, the base handle 235 and the base groove 237. The base protrusion 231 may be protrudently formed at the center position of the lock base 230, as illustrated in FIG. 6A, or it can be protrudently formed at the eccentric position deviated from the center of the lock base 230, as illustrated in FIG. 6b. Further, the base protrusion 231 is formed so as to be accommodated in the lock insertion hole 22 while allowing the pin protrusion 211 to be inserted therein. The lock base 230 may have the protrusion accommodating part 231b formed around the cavity 231a within the lock base 230 so as to prevent unintended separation of the lock pin 210.

As depicted in FIGS. 6A and 6B, the protrusion accommodating part 231b is an inner blocked region of the lock base 230 formed to prevent unintended separation of the inserted and rotated pin protrusion 211 from the lock base 230.

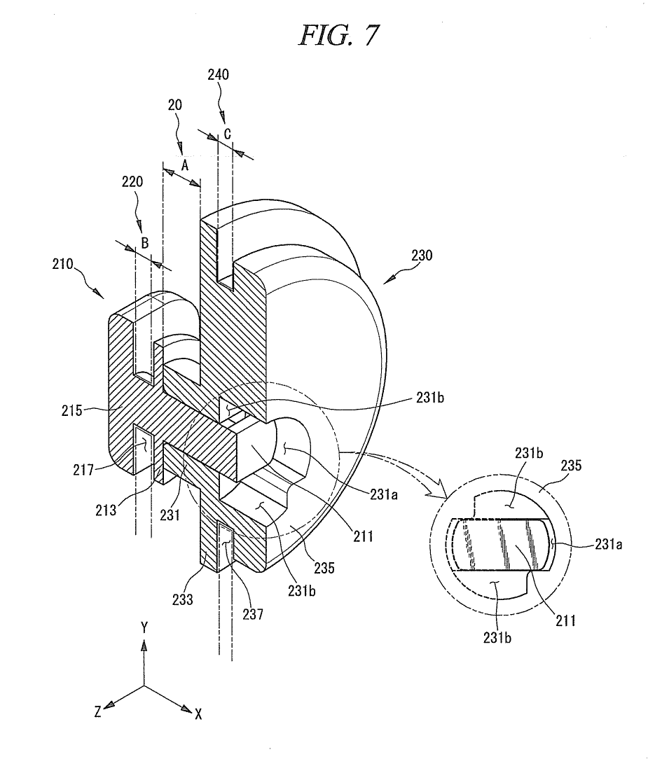

FIG. 7 provides a cross sectional view showing a state immediately after the lock pin 210 is inserted into the lock base 230. FIG. 7 also provides a partial enlarged view of an inserted part.

As illustrated in FIG. 7, the lock pin 210 can be engaged with the lock base 230 as the pin protrusion 211 is inserted into the cavity 231a of the lock base 230. The shield 20 is positioned between the lock pin 210 and the lock base 230 (indicated by an arrow A). Further, the anti-moisture film 210 may be insertion-fitted between the pin supporting plate 213 and the pin handle 215 of the lock pin 210 (indicated by an arrow B). Furthermore, the shield protective film 240 may be fitted between the base supporting plate 233 and the base handle 235 (indicated by an arrow C). As can be seen in FIG. 7, the lock pin 210 and the lock base 230 can be secured to the anti-moisture film 220 and the shield protective film 240, respectively. Thus, either one of the films may be secured to the shield 20 or both of them may be secured to the shield 20. In this way, the anti-moisture film and the shield protective film can be fastened to the inner side and the outer side of the shield 20 and removed therefrom easily when replacement thereof is required.

Further, as shown in the partial enlarged view of FIG. 7 (solid line indicates portions shown in the cross section of FIG. 7, and dotted line indicates the rest lock base and lock pin portions not shown in the cross section of FIG. 7). Immediately after inserted into the lock base 230, the pin protrusion 211 is located on the cavity 231a formed through the base protrusion 231. Accordingly, when the lock pin 210 is moved in an inward direction of the shield 20 (in a direction in which the lock pin is separated from the lock base), the lock pin 210 can be easily separated from the lock base 230 without being interfered by the lock base 230. That is, the lock pin 210 can be easily separated and removed from the lock base 230 just by pulling the lock pin 210 inserted in the lock base 230 in the inward direction again.

FIG. 8 provides a cross sectional view illustrating engagement of the lock pin 210 with the lock base 230 through rotation. FIG. 8 also provides a partial enlarged view of an engaged part.

As shown in FIG. 8, if the lock pin 210 inserted in the lock base 230 is rotated, the pin protrusion 211 is also made to rotate within the lock base 230 in the same direction.

As can be seen from the partial enlarged view of FIG. 8 (solid line indicates portions shown in the cross section of FIG. 8, and dotted line indicates the rest lock base and lock pin portions not shown in the cross section of FIG. 8), the pin protrusion 211 rotated while inserted in the lock base 230 (secured rotation) is moved out of the position on the cavity 231a where insertion and separation is freely possible and then becomes to be positioned on the protrusion accommodating part 231b where separation (of the lock pin from the lock base) is restricted (secured state). As stated above, the protrusion accommodating part 231b is a region formed within the lock base 230 to be located around the cavity 231a of the lock base 230 so as to prevent the movement of the pin protrusion 211 in a direction in which it is separated from the lock base 230. Accordingly, if the pin protrusion 211 comes out of the cavity 231a and is positioned on the protrusion accommodating part 231b (i.e., comes into secured state through secured rotation) as the lock pin 210 rotates while inserted in the lock base 230, the lock pin 210 is engaged with the lock base 230 to be firmly secured thereto. That is, the lock pin 210 (pin protrusion 211) inserted into the lock base 230 is secured to the lock base 230 through secured rotation and can be prevented from being separated or removed from the lock base 230 in unintended manner. In this way, when the pin protrusion 211 of, e.g., the straight line shape is inserted in the cavity 231a having the mutually corresponding shape thereto is rotated within the lock base 230 in secured state, the pin protrusion 211 becomes to come out of the cavity 231a and be positioned on the protrusion accommodating part 231b, thus allowing the lock pin 210 to be firmly fixed in place.

Meanwhile, the user of the helmet 10 may separate the lock pin 210 from the lock base 230 by rotating (unsecure rotating) the lock pin 210 secured to the lock base 230 and thus making the pin protrusion 211 positioned on the cavity 231a again (unsecured state). That is, the lock pin 210 in the secured state is turned into the unsecured state through unsecure rotation. Then, the user of the helmet 10 may pull the unsecured lock pin 210 in the inward direction of the shield 20 and separate the lock pin 210 from the lock base 230 and from the shield 20.

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention.

The scope of the present invention is defined by the following claims rather than by the detailed description of the embodiment. It shall be understood that all modifications and embodiments conceived from the meaning and scope of the claims and their equivalents are included in the scope of the present invention.

What is claimed is:

1. A pin lock device of a shield secured to a helmet main body in an openable/closable manner, the pin lock device comprising:
lock insertion holes, formed through both lateral surfaces of the shield;
lock pins, each being inserted into each of the lock insertion holes from the inside of each lock insertion hole toward the outside thereof; and
lock bases, each being engaged with each of the lock pins from the outside of each lock insertion hole,
wherein each lock pin includes:
a pin protrusion to be fitted into each of the lock bases through each of the lock insertion holes in an outward direction;
a pin supporting plate to be brought into firm contact with each of the lock insertion holes at an inner side of the shield to stop an outward movement of each lock pin; and
a pin handle inwardly extending from the pin supporting plate and serving as a head of each lock pin.

2. The pin lock device of claim 1, wherein each of the lock pins further includes each of pin grooves formed as a curved cut portion between the pin supporting plate and the pin handle and having a cross section smaller than cross sections of the pin supporting plate and the pin handle, and
wherein an anti-moisture film is detachably fitted between the pin grooves along both inner lateral surfaces of the shield.

3. The pin lock device of claim 1, wherein each lock base includes:
a base protrusion to be accommodated in each of the lock insertion holes while allowing each lock pin to be inserted into a cavity formed within the base protrusion;
a base supporting plate to be brought into firm contact with each of the lock insertion holes at an outer side of the shield to stop an inward movement of each lock base; and
a base handle outwardly extending from the base supporting plate and serving as a head of each lock base.

4. The pin lock device of claim 3, wherein each of the lock bases further includes each of base grooves formed as a curved cut portion between the base supporting plate and the base handle and having a cross section smaller than cross sections of the base supporting plate and the base handle, and
wherein a shield protective film is detachably fitted between the base grooves along both outer lateral surfaces of the shield.

5. The pin lock device of claim 4, wherein each lock base further includes a protrusion accommodating part formed within each of the lock bases to be positioned around the cavity to prevent separation of each lock pin from each lock base,
wherein the cavity is formed in a shape corresponding to a shape of the pin protrusion, and
if the pin protrusion inserted in the cavity rotates within each of the lock bases in a secured state, the pin protrusion comes out of the cavity and is positioned on the protrusion accommodation part, and, thus, each lock pin is fixed in place.

6. The pin lock device of claim 5, wherein both the cavity and the pin protrusion have straight line shapes.

7. The pin lock device of claims 4, wherein the base protrusion is formed to be located at an eccentric position of each of the lock bases, and a distance between the lock bases at both lateral surfaces of the shield is varied when each of the lock bases rotates while engaged with each lock pin.

8. A helmet including a shield secured to a helmet main body in an openable/closable manner and pin lock devices fastened to both lateral surfaces of the shield, the helmet comprising:
lock insertion holes, formed through both lateral surfaces of the shield;
lock pins, each being inserted into each of the lock insertion holes from the inside of each lock insertion hole toward the outside thereof; and
lock bases, each being engaged with each of the lock pins from the outside of each lock insertion hole,
wherein each lock pin includes:
a pin protrusion to be fitted into each of the lock bases through each of the lock insertion holes in an outward direction;
a pin supporting plate to be brought into firm contact with each of the lock insertion holes at an inner side of the shield to stop an outward movement of each lock pin; and
a pin handle inwardly extending from the pin supporting plate and serving as a head of each lock pin, and
wherein each lock base includes:
a base protrusion to be accommodated in each of the lock insertion holes while allowing each lock pin to be inserted into a cavity formed within the base protrusion;
a base supporting plate to be brought into firm contact with each of the lock insertion holes at an outer side of the shield to stop an inward movement of each lock base; and
a base handle outwardly extending from the base supporting plate and serving as a head of each lock base.

9. The helmet of claim 8, wherein each of the lock pins further includes each of pin grooves formed as a curved cut portion between the pin supporting plate and the pin handle and having a cross section smaller than cross sections of the pin supporting plate and the pin handle,
wherein an anti-moisture film is detachably fitted between the pin grooves along both inner lateral surfaces of the shield,
wherein each of the lock bases further includes each of base grooves formed as a curved cut portion between the base supporting plate and the base handle and having a cross section smaller than cross sections of the base supporting plate and the base handle, and
wherein a shield protective film is detachably fitted between the base grooves along both outer lateral surfaces of the shield.

10. The helmet of claim 9, wherein each lock base further includes a protrusion accommodating part formed within each of the lock bases to be positioned around the cavity to prevent separation of each lock pin from each lock base,
wherein the cavity is formed in a shape corresponding to a shape of the pin protrusion, and
if the pin protrusion inserted in the cavity rotates within each of the lock bases in a secured state, the pin protrusion comes out of the cavity and is positioned on the protrusion accommodation part, and, thus, each lock pin is fixed in place.

11. The helmet of claim 10, wherein both the cavity and the pin protrusion have straight line shapes.

12. The helmet of claims 9, wherein the base protrusion is formed to be located at an eccentric position of each of the lock bases, and a distance between the lock bases at both lateral surfaces of the shield is varied when each of the lock bases rotates while engaged with each lock pin.
